(19) United States
(12) Patent Application Publication
(10) Pub. No.: US 2007/0172962 A1
(43) Pub. Date: Jul. 26, 2007

(54) Method for measuring gas permeability of any solid permeable material and a device therefor

(76) Inventors: Praveen Kumar Sehgal, Chennai (IN); Janumala Hadassah, Chennai (IN)

Correspondence Address: Richard P. Berg, Esq;c/o LADAS & PARRY, Suite 2100, 5670 Wilshire Boulevard, Los Angeles, CA, 90036-5679, US

(21) Appl. No.: 11/388,225

(22) Filed: Mar. 22, 2006

(30) Foreign Application Priority Data
Jan. 25, 2006 (IN) 230/DEL/2006

(51) Int. Cl. G01N 33/00

(52) U.S. Cl. 436/183

(57) ABSTRACT

An equipment is provided to measure the gas permeability of a solid permeable material. The gas permeated through the material is collected in a solvent and the amount of gas permeated is estimated.

### FIELD OF THE INVENTION

The present invention relates to a method for measuring gas permeability of any solid permeable material and a device therefor. More particularly, the present invention relates to a method and a device to measure the gas (such as oxygen, nitrogen, chlorine, carbondioxide, argon, ethylene) permeability of any solid permeable material. More particularly the present invention provides a method and a device which enables measuring the gas permeability of any solid permeable material accurately and quickly.

The process as well as the device of the present invention is envisaged to have enormous potential applications in the pharmaceutical industry particularly contact lens manufacturers for calculating the accurate oxygen permeability values of the ophthalmic lens, which is an essential requirement for the ophthalmic lens. It has also potential application in measuring the gas permeability of gas masks used for mining as well as other different industrial purposes. In addition, the present invention finds potential application in food processing as well as packaging industry, where the permeability of plastic films to gases like oxygen, ethylene and flavour components is of particular interest. Moreover, the equipment of the present invention has also potential application in the agricultural sector for measuring gas permeability of the materials used in fumigation.

### BACKGROUND OF THE INVENTION

Conventionally, gas permeability of a material is measured in terms of Dk value, which is defined as the intrinsic ability to transport gas. Dk is the volume of gas in ml, passing in 1 second, through the material 1 cm thick and area of 1 Sq cm for every 1 mm Hg partial pressure difference across the material at 35° C. Dk is usually quoted in units of 10⁻¹¹.cm²/sec.mlO₂/ml×mmHg (Fau units).

A good ophthalmic lens should have high oxygen permeability. Ophthalmic lens is used in the treatment of eye disorders, infection in the eye, as bandage lens in traumatised eye to treat dry eyes etc. Oxygen permeability of an ophthalmic lens is a physical property and describes its intrinsic ability to transport oxygen to the cornea, insufficient supply of oxygen results in corneal oedema causing osmotic imbalance. Accurate measurement of oxygen permeability is a very difficult task. There has been a practice of measuring oxygen permeability.

Friedmann et al (Journal of membrane-science, Vol 65, Issues 1-2, pages 93-100, 1992) used the Polarographic method for determination of the permeability coefficients of polymers towards dissolved oxygen in water. The system is characterised by strong stirring on both sides of the membrane to evaluate the boundary layer effect on the permeability. The permeability co-efficients are measured as a function of stirring rate, thickness and the area of the membrane. In the polarographic method the gas permeability (Dk) is calculated with the formula.

$$F = \frac{Dk}{L} Δ P$$

$$Dk = \frac{FL}{Δ P} = \frac{Oxygen flux \times Thickness}{Pressure difference across the membrane}$$

The major limitation associated with this method is that even an error as low as (10 μ) in thickness measurement results in significant error (1 unit) in Dk value. Brennan (Clinical and Experimental Optometry, Vol 70, No 6, 1986) has reported that humidity should be always kept constant, especially for soft lenses. Reference may be made to Ebril et al (Journal of Membrane Science, Vol 26, Issue 2, pages 199-209, 1986) who determined the oxygen permeability coefficients of biomedical membranes by colorimetric method. The method is based on the colorimetric determination of dissolved oxygen in the reduced indigo caramine solution at 700 nm wavelength. The major limitation associated with this device is that the measurement process gets frequently hindered because of stagnant boundary layer formation at the polymer liquid interface in the unstirred cell. Resulting in electrode polarisation and the electrochemical side reactions during measurements. The colorimetric method has thus been found to erroneously exhibit higher permeability values for less permeable polymer membranes.

Compan et al (Biomaterials, 19, 2139-2145, 1998 and Biomaterials, 23, 2767-2772, 2002) reported the use of electrochemical method with a permeometer to measure the oxygen permeability of membranes, employing a cathode consisting of 24 carat gold cylinder and an anode made of silver. The thermistor on the anode monitored the temperature of the experiment. The measurements were carried out with two different conditions eg (1) with no previous treatment of the membrane (2) by performing a process of de-oxygenation of the material by placing on the potentiostatic cell in nitrogen atmosphere. Small electrical current densities in the potentiostatic cell varied with the pH of the solution. The major limitations associated with the method are the following.
a) Electrochemical reactions taking place on the electrode requires constant monitoring of the estimation process,
b) It involves high cost of the equipment, because of using 24 carat gold cathode and silver anode,
c) The experimental technique is very complicated.

### This prompted the researchers to look for a better equipment to measure the oxygen permeability of ophthalmic lens.

Labunda et al (U.S. Pat. No. 6,616,896) reported a system that employs luminescence quenching to produce a liquid indicative of oxygen concentration. The device includes an airway adapter, sampling cell having a sensor that is excited into luminescence reflection of the concentration of oxygen in gases flowing through the airway. The main draw back of such systems is that the sensors require delicate handling. The reflection of luminescence through the airway, to measure the permeability of gas may not give accurate results. The fabrication of the sensor is very difficult and requires skilled manpower; the measurement requires constant monitoring and is time consuming.

Akira et al (Japan patent JP 6273309) reported the use of a highly sensitive apparatus to measure the gas permeability. This is done by sealing a measuring equipment hermitically, while bringing a barrier resin film on one side thereof into contact with a volatile liquid and taking out a gas compound permeated through the barrier resin film from the other side using an inert gas fed by a predetermined quantity. The ratio between liquid phase and gas phase on the contact phase of the film requires to be adjusted. The measuring of gas permeation through the resin films requires sophisticated sensors, wherein the measurement should be done with many fine adjustments and requires constant monitoring. The major limitation associated with the equipment is that it can be used to measure the gas permeability in vivo, and cannot be effectively used for all types of materials.

### OBJECTS OF THE INVENTION

The main object of the present invention is to provide a method for measuring gas permeability of any solid permeable material and a device therefor, which obviates the limitations stated above.

Another object of the present invention is to provide a device which measures the gas permeability of any solid permeable material accurately.

Yet another object of the present invention is to provide a device which can measure the gas permeability of any solid permeable material with different thickness and radius of curvature.

Still another object of the present invention is to provide a device which can measure the gas permeability of collagen bandage lenses in wet and dry conditions.

Yet another object of the present invention is to measure the gas permeability by simple methods like titration, colorimetric analysis.

Still another object of the present invention is that it can measure the permeability of other gases like oxygen, nitrogen, chlorine, carbondioxide, argon, ethylene.

In the drawing accompanying this specification

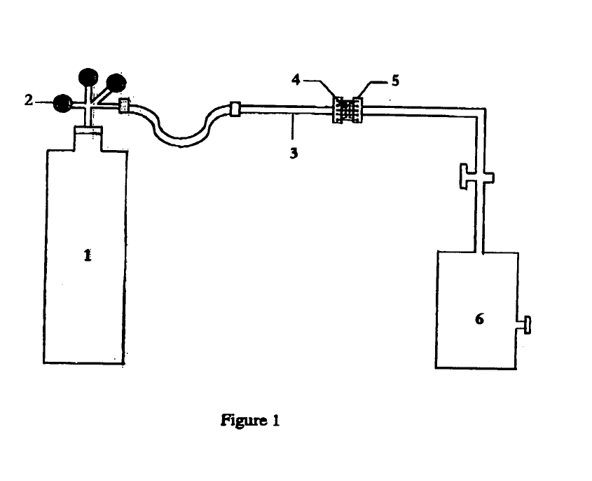

FIG. 1 represents a schematic diagram of the gas permeability equipment

Different components of FIG. 1 are as follows:
1. refers to gas source
2. refers to pressure control valve
3. refers to piping
4. refers to sample holder
5. refers to rings with nuts and bolts
6. refers to solvent container

Accordingly, the present invention provides a method for measuring gas permeability of any solid permeable material, which comprises:
i. impinging gas on one surface of the solid permeable material, at a pressure in the range of 5 mmHg to 25 mmHg., collecting the so permeated gas at the surface of the said material and estimating the quantity of the said permeated gas captured by a known solvent,
ii. removing the said material from the sample holder, closing the gap between the two sides of the said sample holder and passing gas at the said pressure as above and estimating the gas captured in the same solvent as used in step (i),
iii. estimating the difference in values as obtained in step (i) and step (ii) to obtain the amount of gas dissolved in the solvent, followed by calculating the gas permeability by known method to obtain gas permeability of the material.

In an embodiment of the present invention, the gas used may be such as oxygen, nitrogen, chlorine, carbondioxide, argon, ethylene.

In another embodiment of the present invention, the solid permeable material used may be such as ophthalmic lenses, plastic films, dry collagen bandage lens, wet collagen bandage lens, resin films, PVP polymeric films, PVA polymeric films, collagen sponge.

In yet another embodiment of the present invention, the solvent used for collecting the permeated gas may be selected from ethanol, acetone, carbon tetra chloride, water.

In still another embodiment of the present invention, the estimation of the permeated gas dissolved in the solvent may be carried out by known methods such as titration, colorimetric analysis.

Accordingly, the present invention provides an equipment for measuring gas permeability of any solid permeable material using the method as herein under described, which comprises: a known gas source (1) capable of supplying gas at a pressure in the range of 5 mm Hg to 25 mm Hg, the said gas source being connected through means such as piping (3) to one end of a sample holder (4), optionally incorporated with cushion (not in diagram), enabling impingement of the said gas on to one surface of the material, the other end of the said sample holder (4) being connected through means such as piping to a solvent container (6).

In an embodiment of the present invention, the gas piping may be provided with control valves.

In another embodiment of the present invention, the means for measuring the gas pressure may be such as manometer, electrical, electronic, mechanical device.

In yet another embodiment of the present invention, the gas piping between the gas source and the sample holder may be provided with a gas control valve.

### DESCRIPTION OF THE PRESENT INVENTION

The equipment consists of a gas source (1) with pressure control valve (2) that is connected by piping (3) to a sample holder (4), which is optionally incorporated with cushion (not in diagram) to facilitate putting curved lenses. The other end of the sample holder is connected to a solvent container (6) with a piping. The sample holder consists of polymeric rings with nuts and bolts to keep the sample in place. The equipment measures the gas permeability of any solid permeable material. Initially, the equipment is made free from other gases by creating vacuum in the chamber by connecting to a vacuum pump. Thickness and area of the material are measured. The material is placed in between the sample holder (5). Known solvent is taken in the solvent container and gas is released from gas source for a period in the range of 5 sec. to 20 sec. The pressure is maintained in the range of 5 mm to 20 mmHg. The permeated gas dissolves in the solvent. The solvent with dissolved gas is taken separately and the amount of gas passing through the material is estimated by a known method. This gives the experimental value x₂. The entire experiment is carried out at a temperature in the range of 25° C.-35° C. The material is removed from the sample holder and the experiment is repeated. The solvent with the dissolved gas without the material gives the control value x₁. The difference in the experimental value and the control gives the amount of dissolved gas in the solvent (x₂-x₁). The gas permeability of the material is then calculated by using the following formula and is expressed as Dk value of the sample.

$$Gas permeability ( Dk ) = \frac{760 \times 22.4 ( {x}_{2} - {x}_{1} )}{m \times 273 \times {t}_{1}} \times \frac{{T}^{2}}{a \times {p}_{i}}$$

### Where,

Amount of dissolved gas=(x₂−x₁) mg of gas
T₂=Thickness of the material
t₁=Temperature in ° C.
a=Area of the material
p₁=Experimental pressure
m=Molecular weight of the gas

The inventive step of the process lies firstly in providing a simple and economic method for capturing the permeated gas in a solvent followed by measuring the gas so permeated by methods such as titration, colorimetric analysis.

Secondly, it resides lies in the combination assembly of a gas source, sample holder, and a container having solvent like ethanol, acetone, carbon tetrachloride, water and also the control means for gas passage for measuring the permeated gas.

The following examples are given by way of illustration only and therefore should not be construed to limit the scope of the present invention.

### EXAMPLE 1

The equipment was made free from other gases by creating vacuum in the chamber by connecting to a vacuum pump. Thickness and area of the ophthalmic lens made of Hydroxy ethy methyl acrylate (HEMA) was measured and found to be 0.05 mm and 1.76 sq cm. The lens was then inserted in the sample holder (5). 25 ml of ethanol was taken in the solvent container and closed with the stopcock without letting in air. The oxygen gas was released from the cylinder (1) for 10 sec. The pressure of the gas entering the lens was monitored to be 10 mmHg. The oxygen permeated through the lens was collected in ethanol. The entire experiment was carried out at 35° C.

The amount of oxygen dissolved in ethanol was measured by titration using following steps. In the first step 0.1 ml of manganese sulphate solution (48%) was added to the solvent and mixed carefully, without letting in air. Then 0.2 ml alkaline potassium iodide (i.e. KI 15% in KOH 70%) mixed together was added. A pink brown precipitate was formed. At that point it was set aside for a while, 0.3 ml of 50% H₂SO₄ was added and mixed thoroughly and allowed to stand for 2 minutes. The precipitate dissolved. 10 ml of the sample was transferred to a conical flask and few drops of starch was added. The sub sample was titrated against 0.31% sodium thio-sulphate solution, the solution turned clear. The experimental value, with the dry collagen bandage lens from the above titrate was 6.7 ml. Each 1 ml of thio-suplhate titrate was equivalent to 0.1 mg of oxygen in the 10 ml sub sample that is 0.67 mg of oxygen was dissolved in the sub sample.

The lens was removed from the sample holder and the equipment was made free from other gases. The experiment was repeated as above and the amount of oxygen dissolved was measured by titration. The control value of oxygen without any lens was 4.7 ml that is 0.47 mg of oxygen. The difference in the values of the titrate 0.67−0.47=0.20 mg of oxygen. Taking the above parameters into consideration the oxygen permeability was calculated from the formula. Oxygen permeability of the ophthalmic lens (HEMA) was found to be 30×10⁻¹¹ Cm²/Sec. ml O₂/ml×mm Hg Fatt units.

### EXAMPLE 2

In this method, the equipment was made free from other gases by creating vacuum in the chamber by connecting to a vacuum pump. Thickness and area of the dry collagen bandage lens were measured and found to be 0.05 mm and 1.76 sq cm. The lens was then inserted in the sample holder (5) and tightened. 25 ml of ethanol was taken in the solvent container and was closed with the stopcock without letting in air. The oxygen gas was released from the cylinder (1) for 10 sec. The pressure of the gas entering the membrane was monitored to be 10 mmHg. The oxygen permeated through the lens was collected in enthanol. The entire experiment was carried out at 35° C.

The amount of oxygen dissolved in ethanol was measured by titration. In the first step 0.1 ml of manganese sulphate solution (48%) was added to the solvent and mixed carefully, without letting in air. Then 0.2 ml alkaline potassium iodide (i.e. KI 15% in KOH 70%) mixed together was added. A pink brown precipitate was formed. At that point it was set aside for a while, 0.3 ml of 50% H₂SO₄ was added and mixed thoroughly and allowed to stand for 2 minutes. The precipitate dissolved.

10 ml of the sample was transferred to a conical flask and few drops of starch was added. The sub sample was titrated against 0.31% sodium thio-sulphate solution, the solution turned clear. The experimental value, with the dry collagen bandage lens from the above titrate was 6.5 ml. Each 1 ml of thio-suplhate titrate was equivalent to 0.1 mg of oxygen in the 10 ml sub sample that is 0.65 mg of oxygen was dissolved in the sub sample.

The lens was removed from the sample holder and the equipment was made free from other gases by connecting to a vacuum pump. The experiment was repeated as above and the amount of oxygen dissolved was measured by titration. The control value of oxygen without any lens was 4.7 ml that is 0.47 mg of oxygen. The difference in the values of the titrate 0.65−0.47=0.18 mg of oxygen. Taking the above parameters into consideration the oxygen permeability was calculated with the formula as shown above.

Oxygen permeability (Dk value) of dry collagen bandage lens was found to be 26×10⁻¹¹ Cm²/Sec. ml O₂/ml×mm Hg Fatt units.

### EXAMPLE 3

The equipment was made free from other gases by creating vacuum in the chamber by connecting to a vacuum pump. The thickness and area of the wet collagen bandage lens to be tested for oxygen permeability were measured and found to be 0.06 mm and 1.76 sq cm. The lens was then inserted in the sample holder and it was tightened. 28 ml of ethanol was taken in the solvent container and was closed with the stopcock without letting in air. The oxygen gas was released from the cylinder for 10 sec. The pressure of the gas entering the membrane was monitored to be 10 mmHg. The oxygen permeated through the lens was collected in ethanol. The amount of oxygen dissolved in ethanol was measured by titration.

In the first step 0.1 ml of manganese sulphate solution (48%) was added and mixed carefully, without letting in air. Then 0.2 ml alkaline potassium iodide (e.g. KI 15% in KOH 70%) mixed together was added. A pink brown precipitate was formed. At that point it was set aside for a while, 0.3 ml of 50% H₂SO₄ was added and mixed thoroughly and allowed to stand for 2 minutes. The precipitate dissolved.

10 ml of the sample was transferred to a conical flask and few drops of starch was added. The subsample was titrated against 0.31% sodium thio-sulphate solution, the solution turned clear. The experimental value, with the wet collagen bandage lens from the above titrate was 6.7 ml that is 0.67 mg of oxygen as each 1 ml of thio-suplhate titrate was equivalent to 0.1 mg of oxygen in the 10 ml sub sample. The lens was removed from the lens holder and the equipment was made free from other gases by connecting to a vacuum pump. The experiment was repeated as above and the amount of oxygen dissolved was measured by titration. The control value of oxygen without any lens as 4.7 ml that is 0.47 mg of oxygen as 1 ml of thiosulphate titrate is equivalent to 0.1 mg of oxygen in the 10 ml sub sample. The difference in the values of the titrate 0.67−0.47=0.20 mg of oxygen. The experiment was performed at 35° C. The Dk was calculated with the formula as shown above.

The oxygen permeability (Dk) of wet collagen bandage lens was found to be 28×10⁻¹¹ Cm²/Sec. ml O₂/ml×mm Hg Fatt units.

### EXAMPLE 4

The equipment was made free from other gases by creating vacuum in the chamber by connecting to a vacuum pump. The lens parameters like thickness and area of the ophthalmic lens made of PMMA (poly methyl methacrylate) to be tested for oxygen permeability were measured and found to be 0.06 mm and 1.76 sq cm. The lens was then inserted in the sample holder. 25 ml of ethanol was taken in the solvent container and was closed with the stopcock without letting in air. The oxygen gas released from the cylinder for 10 sec. The pressure of the gas entering the membrane was monitored to be 10 mmHg. The oxygen permeated through the lens was collected in ethanol. The amount of oxygen dissolved in ethanol was measured by titration.

In the first step 0.2 ml of manganese sulphate solution (48%) was added and mixed carefully, without letting in air. Then 0.3 ml alkaline potassium iodide (i.e. KI 15%) in KOH 70%) mixed together was added. A pink brown precipitate was formed. At this point it was set aside for a while, 0.4 ml of 50% H₂SO₄ was added and mixed thoroughly and allowed to stand for 2 minutes. The precipitate dissolved 10 ml of the sample was transferred to a conical flask and few drops of starch were added. The sub sample was titrated against 0.31% sodium thio sulphate solution, the solution turned clear. The experimental value was 5.3 ml that is 0.53 mg of oxygen as each 1 ml of thio-suplhate titrate is equivalent to 0.1 mg of oxygen in the 10 ml sub sample. A control without any lens was carried out with the same process as above. The control value of oxygen without any lens was 4.7 ml that is 0.47 mg of oxygen. The difference in the values of the titration gives the amount of oxygen that is dissolved in the solvent that is 0.06 mg of oxygen. Dk was calculated from the formula

Dk of PMMA ophthalmic lens found to be 19×10⁻¹¹ Cm²/Sec. ml O₂/ml×mm Hg Fatt units.

### EXAMPLE 5

The equipment was made free from other gases by creating vacuum in the chamber by connecting to a vacuum pump. The thickness and area of the polyvinyl alcohol film to be tested for chlorine permeability were measured and found to be 0.05 mm and 1.76 sq cm. The film was then inserted in the lens holder. 250 ml of ethanol was taken in the solvent container and was closed with the stopcock without letting in air. The chlorine gas was released from the cylinder for 10 sec. The pressure of the gas entering the film was monitored to be 10 mmhg. The gas permeated through the film was collected in the solvent. The amount of chlorine gas dissolved in the solvent was measured by colorimeter.

5 flasks were taken and 50 ml of the solvent is transferred to each flask. 5 ml of 5% ethanoic acid was added to each flask. To this 1 ml of 0.5% Milton sterilising fluid was added to flask 2, 2 ml was added to flask 3, 4 ml was added to flask 4 and 8 ml was added to flask 5. 5 ml of 2% potassium iodide (KI) was added to all the flask. The content were mixed thoroughly and allowed to stand for 5 minutes for colour to develop. The colour intensity was measured at 440 nm with a colorimeter. The readings were flask 1=0.00, flask 2=0.06, flask 3=0.12, flask 4=0.24, and flask 5=0.43. A graph between absorbance and concentration was plotted. The point of incidence is taken as the average value and was multiplied with 71/74.5=0.11 mg of the chlorine. The chlorine gas permeability of polyvinyl alcohol film was found to be 12×10⁻¹¹ Cm²/Sec. ml O₂/ml×mm Hg Fatt units from the formula as shown above.

### EXAMPLE 6

The equipment was made free from other gases by creating vacuum in the chamber by connecting to a vacuum pump. The thickness and area of the ophthalmic lens to be tested for nitrogen permeability were measured and found to be 0.05 mm and 1.76 sq cm. The lens was then inserted in the sample holder. 30 ml of carbon tetrachloride was taken in the solvent container and was closed with the stopcock without letting in air. The nitrogen gas was released slowly from the cylinder for 10 sec. The pressure of the gas entering the membrane was monitored to be 10 mmHg. The gas permeated through the lens was collected in carbon tetra chloride. The amount of nitrogen gas dissolved in the solvent was measured by analytical estimation. The flask was made air tight so as not to allow any other gas to interfere with the solvent. The weight of the solvent container was measured and was 23.002 gms and this was taken as the experimental value. The experiment was repeated without any lens in the lens holder. The control value was 23.00 gms. The difference in values as obtained in step (i) and step (ii) to obtain the gas permeability value of the ophthalmic lens, that is 0.002 gms is equal to 0.2 mg.Dk was calculated from the formula as shown above, the Dk of ophthalmic lens was found to 28×10⁻¹¹ Cm²/Sec. ml O₂/ml×mm Hg Fatt units.

### EXAMPLE 7

The equipment was made free from other gases by creating vacuum in the chamber by connecting to a vacuum pump. The thickness and area of the collagen sponge material to be tested for oxygen permeability were measured and found to be 0.05 mm and 1.76 sq cm. The material was then inserted in the sample holder and it was tightened 30 ml of ethanol was taken in the solvent container and was closed with the stopcock without letting in air. The oxygen gas was released slowly from the cylinder for 10 sec. The pressure of the gas entering the membrane was monitored to be 10 mmHg. The oxygen permeated through the lens was collected in ethanol. The amount of oxygen dissolved in ethanol was measured by titration.

In the first step 0.2 ml of manganese sulphate solution (48%) was added and mixed carefully, without letting in air. Then 0.3 ml alkaline potassium iodide (i.e. KI 15% in KOH 70%) mixed together was added. A pink brown precipitate was formed. At this point it was set aside for a while, 0.4 ml of 50% H₂SO₄ was added and mixed thoroughly and allowed to stand for 2 minutes. The precipitate dissolved 10 ml of the sample was transferred to a conical flask and few drops of starch were added. The sub sample was titrated against 0.31% sodium thio sulphate solution, the solution turned clear. The experimental value was 5.3 ml that is 0.53 mg of oxygen as each 1 ml of thio-suplhate titrate is equivalent to 0.1 mg of oxygen in the 10 ml sub sample. A control without any material was carried out with the same process as above. The control value of oxygen without lens was carried out with the same process as above. The control value of oxygen without any lens was 4.7 ml that is 0.47 mg of oxygen. The difference in the values of the titration gives the amount of oxygen that is dissolved in the solvent (ethanol). The difference in the titrate values was 0.06 mg of oxygen.

10 ml of the sample was transferred to a conical flask and few drops of starch was added. The sub sample was titrated against 0.31% sodium thio-sulphate solution, the solution turned clear. The experimental value, with sponge material from the above titrate was 0.62 mg of oxygen. Each 1 ml of thio-suplhate titrate is equivalent to 0.1 mg of oxygen in the 10 ml sub sample. The control value of oxygen without any lens was 5.0 ml that is 0.50 mg of oxygen. The difference in the titrate values was 0.18 mg of oxygen. Dk of collagen sponge material was found to be 23×10⁻¹¹ Cm²/Sec. ml O₂/ml×mm Hg Fatt units.

### EXAMPLE 8

The equipment was made free from other gases by creating vacuum in the chamber by connecting to a vacuum pump. The resin material parameters like thickness and area to be tested for carbondioxide gas permeability were measured and found to be 0.07 mm and 2.00 sq.cm. The material was then inserted in the sample holder. 25 ml of water was taken in the solvent container and was closed with the stopcock without letting in air. The carbondioxide gas was released from the cylinder for 10 sec. The pressure of the gas entering the material was monitored to be 10 mmHg. The gas permeated through the material was collected in water. The amount of gas dissolved in water was measured by titration.

In the first step 0.2 ml of manganese sulphate solution (48%) was added and mixed carefully, without letting in air. Then 0.3 ml alkaline potassium iodide (i.e. KI 15% in KOH 70%) mixed together was added. A pink brown precipitate was formed. At this point it was set aside for a while, 0.4 ml of 50% H₂SO₄ was added and mixed thoroughly and allowed to stand for 2 minutes. The precipitate dissolved 10 ml of the sample was transferred to a conical flask and few drops of starch were added. The sub sample was titrated against 0.31% sodium thio sulphate solution, the solution turned clear. The experimental value was 5.3 ml that is 0.53 mg of carbon dioxide gas as each 1 ml of thio-suplhate titrate is equivalent to 0.1 mg of carbondioxide gas in the 10 ml sub sample. A control without any material was carried out with the same process as above. The control value of carbondioxide gas without any material was 4.7 ml that is 0.47 mg of carbondioxide. The difference in the values of the titration gives the amount of carbondioxide gas that is dissolved in the solvent that is 0.06 mg of carbondioxide. Dk was calculated from the formula

Dk of resin material was found to be 19×10⁻¹¹ Cm²/Sec. ml O₂/ml×mm Hg Fatt units.

### EXAMPLE 9

The equipment was made free from other gases by creating vacuum in the chamber by connecting to a vacuum pump. The thickness and area of the poly vinyl pyrrolidone film to be tested for oxygen permeability were measured and found to be 0.05 mm and 1.76 sq cm. The lens was then inserted in the sample holder and its was tightened 30 ml of ethanol was taken in the solvent container and was closed with the stopcock without letting in air. The oxygen gas was released slowly from the cylinder for 10 sec. The pressure of the gas entering the membrane was monitored to be 10 mmHg. The oxygen permeated through the lens was collected in ethanol. The amount of oxygen dissolved in ethanol was measured by titration.

In the first step 0.2 ml of manganese sulphate solution (48%) was added and mixed carefully, without letting in air. Then 0.3 ml alkaline potassium iodide (i.e. KI 15% in KOH 70%) mixed together was added. A pink brown precipitate was formed. At this point it was set aside for a while, 0.4 ml of 50% H₂SO₄ was added and mixed thoroughly and allowed to stand for 2 minutes. The precipitate dissolved 10 ml of the sample was transferred to a conical flask and few drops of starch were added. The sub sample was titrated against 0.31% sodium thio sulphate solution, the solution turned clear. The experimental value was 5.3 ml that is 0.53 mg of oxygen as each 1 ml of thio-suplhate titrate is equivalent to 0.1 mg of oxygen in the 10 ml sub sample. A control without any lens was carried out with the same process as above. The control value of oxygen without any lens was 4.7 ml that is 0.47 mg of oxygen. The difference in the values of the titration gives the amount of oxygen that is dissolved in the solvent (ethanol). The difference in the titrate values was 0.06 mg of oxygen.

10 ml of the sample was transferred to a conical flask and few drops of starch was added. The sub sample was titrated against 0.31% sodium thio-sulphate solution, the solution turned clear. The experimental value, with polyvinyl pyrrolidone film from the above titrate was 0.62 mg of oxygen. Each 1 ml of thio-suplhate titrate is equivalent to 0.1 mg of oxygen in the 10 ml sub sample. The control value of oxygen without any lens was 4.7 ml that is 0.47 mg of oxygen. The difference in the titrate values was 0.15 mg of oxygen. Dk of polyvinyl pyrrolidone film was found to be 22×10⁻¹¹ Cm²/Sec. ml O₂/ml×mm Hg Fatt units.

### EXAMPLE 10

The equipment was made free from other gases by creating vacuum in the chamber by connecting to a vacuum pump. The parameters of the plastic film like thickness and area of the film to be tested for argon gas permeability were measured and found to be 0.07 mm and 2.00 sq.cm. The material was then inserted in the sample holder. 25 ml of water was taken in the solvent container and was closed with the stopcock without letting in air. The argon gas was released from the cylinder for 10 sec. The pressure of the gas entering the material was monitored with an electrical manometer to be 10 mmHg. The gas permeated through the material was collected in water. The amount of gas dissolved in water was measured by titration.

In the first step 0.2 ml of manganese sulphate solution (48%) was added and mixed carefully, without letting in air. Then 0.3 ml alkaline potassium iodide (i.e. KI 15% in KOH 70%) mixed together was added. A pink brown precipitate was formed. At this point it was set aside for a while, 0.4 ml of 50% H₂SO₄ was added and mixed thoroughly and allowed to stand for 2 minutes. The precipitate dissolved 10 ml of the sample was transferred to a conical flask and few drops of starch were added. The sub sample was titrated against 0.31% sodium thio sulphate solution, the solution turned clear. The experimental value was 5.3 ml that is 0.53 mg of argon gas as each 1 ml of thio-suplhate titrate is equivalent to 0.1 mg of oxygen in the 10 ml sub sample. A control without any material was carried out with the same process as above. The control value of argon gas without any film was 4.7 ml that is 0.47 mg of argon gas without any film was 4.7 ml that is 0.47 mg of argon. The difference in the values of the titration gives the amount of argon gas that is dissolved in the solvent that is 0.06 mg of argon. Dk was calculated from the formula

Dk of plastic film was found to be 19×10⁻¹¹ Cm²/Sec. ml O₂/ml×mm Hg Fatt units.

### EXAMPLE 11

The equipment was made free from other gases by creating vacuum in the chamber by connecting to a vacuum pump. The parameters of the plastic film like thickness and area of the film to be tested for ethylene gas permeability were measured and found to be 0.06 mm and 1.80 sq.cm. The material was then inserted in the sample holder. 25 ml of water was taken in the solvent container and was closed with the stopcock without letting in air. The ethylene gas was released from the cylinder for 10 sec. The pressure of the gas entering the material was monitored with a manometer to be 10 mmHg. The gas permeated through the material was collected in water. The amount of gas dissolved in water was measured by titration in the first step 0.2 ml of manganese sulphate solution (48%) was added and mixed carefully, without letting in air. Then 0.3 ml alkaline potassium iodide (i.e. KI 15% in KOH 70%) mixed together was added. A pink brown precipitate was formed. At this point it was set aside for a while, 0.4 ml of 50% H₂SO₄ was added and mixed thoroughly and allowed to stand for 2 minutes. The precipitate dissolved 10 ml of the sample was transferred to a conical flask and few drops of starch were added. The sub sample was titrated against 0.31% sodium thio sulphate solution the solution turned clear. The experimental value was 5.2 ml that is 0.52 mg of ethylene gas as each 1 ml of thio-suplhate titrate is equivalent to 0.1 mg of oxygen in the 10 ml sub sample. A control without any material was carried out with the same process as above. The control value of ethylene gas without any film was 4.7 ml that is 0.47 mg of ethylene gas without any film was 4.7 ml that is 0.47 mg of ethylene. The difference in the values of the titration gives the amount of ethylene gas that is dissolved in the solvent that is 0.05 mg of ethylene. Dk was calculated from the formula

Dk of plastic film was found to be 18×10⁻¹¹ Cm²/Sec. ml O₂/ml×mm Hg Fatt units

The main advantages of the present invention are:
1. The equipment comprises of simple glassware, which is very cost effective, it can be assembled and dissembled without any problem compared to the sophisticated equipments, which are in use.
2. The equipment measures the gas permeability of any solid permeable membrane with different thickness.
3. The equipment measures the gas permeability, of all types of ophthalmic lenses with different thickness and radius of curvature.
4. The equipment measures the gas permeability of collagen bandage lens in wet and dry conditions
5. The equipment measures the gas permeability of any solid permeable material very accurately and in less time.
6. The gas transmissibility and equivalent gas percentage of any solid permeable material can be obtained from the gas permeability values.

We claim:

1. A method for measuring gas permeability of any solid permeable material which comprises:
i. impinging gas on one surface of the material, at a pressure in the range of 5 mmHg to 25 mmHg, collecting the said permeated gas at the surface of the said material and estimating the quantity of the said permeated gas captured in the solvent,
ii. removing the said material from the sample holder, closing the gap between the two sides of the said sample holder and passing gas at the said pressure as above and estimating the gas captured in the solvent,
iii. estimating the difference in values as obtained in step (i) and step (ii) to obtain the amount of gas dissolved in the solvent.

2. A method as claimed in claim 1, wherein the gas used is selected from the group comprising of oxygen, nitrogen, chlorine, carbon dioxide, argon, ethylene.

3. A method as claimed in claims 1 and 2, wherein the solid permeable material used is selected from the group comprising of ophthalmic lenses, plastic films, dry collagen bandage lens, wet collagen bandage lens, resin films, PVP polymeric films, PVA polymeric films, collagen sponge.

4. A method as claimed in claims 1 to 3, wherein solvent used for collecting the permeated gas is selected from ethanol, acetone, carbon tetra chloride, water.

5. A method as claimed in claims 1 to 4, wherein the estimation of the permeated gas dissolved in the solvent is carried out by known methods such as titration, colorimetric analysis.

6. A device for measuring gas permeability of any solid permeable material using the method such as herein described, the said equipment comprising: a gas source (1) capable of supplying gas at a pressure in the range of 5 mm Hg to 25 mm Hg, the said gas source being connected through piping means (3) to one end of a sample holder (4), optionally incorporated with a cushion, enabling impingement of the said gas on to one surface of the material, the other end of the said sample holder (4) being connected through piping means to a solvent container (6).

7. A device as claimed in claim 6, wherein the gas piping is provided with control valves.

8. A device as claimed in claims 6 and 7, wherein the means for measuring the gas pressure is such as manometer, electrical, electronic, mechanical device.

9. A device as claimed in claims 6 to 8, wherein the gas piping between the gas source and the sample holder is provided with a gas control valve.

10. A method for measuring gas permeability of any solid permeable material and a device therefor, substantially as herein described with reference to the examples and drawing accompanying the specification.
